(19) United States
(12) Patent Application Publication
(10) Pub. No.: US 2026/0152454 A1
(43) Pub. Date: Jun. 4, 2026

(54) PROCESS FOR PRODUCING MONOMERS

(71) Applicant: UOP LLC, Rosemont, IL (US)

(72) Inventors: Jan de Ren, Arlington Heights, IL (US); John Anthony Petri, Wauconda, IL (US); Steven Francis Zink, Batavia, IL (US); Robert Szczesniak, Chicago, IL (US); Ping Sun, Hinsdales, IL (US)

(21) Appl. No.: 19/407,999

(22) Filed: Dec. 3, 2025

Related U.S. Application Data
(60) Provisional application No. 63/727,895, filed on Dec. 4, 2024.

(51) Int. Cl. C07C 5/32; C07C 7/10; C07C 51/16; C07C 67/39; C10G 65/12

(52) U.S. Cl. CPC C07C 5/32; C07C 7/10; C07C 51/16; C07C 67/39; C10G 65/12

(57) ABSTRACT

A process for producing monomers is disclosed. The process comprises hydrocracking a pyrolysis oil stream in a hydrocracking reactor over a hydrocracking catalyst in the presence of a hydrocracking hydrogen stream at hydrocracking conditions to open rings present in said pyrolysis oil stream to produce a hydrocracked effluent stream. The hydrocracked effluent stream is fractionating in a fractionation column to provide a naphtha stream. The naphtha stream is contacted with a NEP catalyst and hydrogen to produce a light paraffinic stream or with a reforming catalyst to produce an aromatics stream.

### FIELD

The field is related to a process producing monomers. Particularly, the field is related to producing monomers from a plastic feed.

### BACKGROUND

Recycling is the process of collecting and processing materials from waste streams that would otherwise be disposed of as trash and turning them into new products. Recycling has benefits for communities and for the environment, since it reduces the amount of waste sent to landfills, conserves natural resources such as timber, water, and minerals, increases economic security by tapping a domestic source of materials, prevents pollution by reducing the need to collect new raw materials, and saves energy. After collection, recyclables may be sent to a material recovery facility (“MRF”) to be sorted, cleaned, and processed into useable materials.

The recovery and recycling of waste plastics is held with deep interest by the general public which has been participating in the front end of the process for decades. Past plastic recycling paradigms can be described as mechanical recycling. Mechanical recycling entails sorting, washing and melting recyclable plastic articles into molten plastic materials to be remolded into a new clean article. However, this mechanical recycling process has not proven economical. The melting and remolding paradigm has encountered several limitations, including economic and qualitative. Collection of recyclable plastic articles at materials recovery facilities inevitably includes non-plastic articles that had to be separated from the recyclable plastic articles. Similarly, collected articles of different plastics have to be separated from each other before undergoing melting because the articles molded of different plastics would not typically have the quality as an article molded of the same plastic. Sorting of collected plastic articles from non-plastic articles and then into like plastics adds expense to the process that reduces its economic efficiency. Additionally, recyclable plastic articles have to be properly cleaned to remove non-plastic residues before melting and remolding which additionally increases the expense of the process. The recovered plastic also does not possess the quality of virgin grade plastic resins. The economics of the plastic recycling process and the lower quality of recycled plastic have prevented widespread acceptance of this renewable resource.

A paradigm shift has enabled the chemical industry to rapidly respond with new chemical recycling processes for waste plastics. The new paradigm is to chemically convert the recyclable plastics in a pyrolysis process operated at about 350 to 600° C. to liquids. The liquids can be refined in a refinery to fuels, petrochemicals and even monomers that can be re-polymerized to make virgin plastic resins.

Plastics are long-lasting chemicals and can accumulate in the environment in landfills, oceans. Plastics such as polyethylene, polypropylene, polyvinylchloride, polyethylene terephthalate, polystyrene and others comprise polyolefins and aromatics-based monomers. These plastics can be reduced in molecular weight using a thermal process such as pyrolysis for recycling plastics. However, the resultant molecular weight of the recycled plastics comprises primarily carbon numbers 8 through 20. The recycled plastics simply have not been converted back to virgin plastics to complete the plastics circularity loop with the full carbon dioxide sequestration.

Conversion of plastics back to monomers presents a circular way of recycling a renewable resource that as of yet has not been fully economically developed. What is needed is a viable process to convert plastic articles directly back to monomers.

### BRIEF SUMMARY

A process for producing monomers is disclosed. The process comprises hydrocracking a pyrolysis oil stream in a hydrocracking reactor over a hydrocracking catalyst in the presence of a hydrocracking hydrogen stream at hydrocracking conditions to open rings present in said pyrolysis oil stream to produce a hydrocracked effluent stream. The hydrocracked effluent stream is fractionated in a fractionation column to provide a naphtha stream. The naphtha stream is contacted with a NEP catalyst and hydrogen to produce a light paraffinic stream and/or with a reforming catalyst to produce an aromatics stream. The present process converts recycled plastics back into virgin plastics, which sequesters carbon dioxide.

The process converts the plastics into lighter paraffins of carbon number 10 and lower, and more preferentially into light paraffins of carbon number 6 and lower. This further reduction in carbon number to 10 and lower or 6 and lower is performed by a hydrocracking process. The lower carbon number paraffins can be a feed source for a catalytic naphtha cracking process, such as the NEP process. The NEP process may further reduce the light paraffins into ethane and propane. The ethane and propane from the NEP process may be processed in a steam cracker to produce ethylene and propylene. Alternatively, the propane may be a feed to a propane dehydrogenation unit to produce propylene. The olefins are converted into virgin plastics through a polymerization process. Plastics are a product that sequester carbon dioxide. In another embodiment, carbon numbers comprising C₇ through C₁₀ hydrocarbons from the hydrocracking process and the recycled plastics may be processed in a catalytic reforming unit. The catalytic reforming unit produces aromatics that are processed in a downstream complex of units that convert these aromatics into paraxylene or xylene isomers. The paraxylene is converted into purified terephthalic acid (PTA). The PTA is converted into polyethylene terephthalate (PET), a plastic. In another embodiment, C₆ and lighter paraffins may be fed to the NEP process and C₇ through C₁₀ paraffins may be fed to a catalytic reforming unit.

### BRIEF DESCRIPTION OF THE DRAWINGS

FIG. 1 is a schematic drawing of an exemplary embodiment of the process for producing monomers.

FIG. 2 is a schematic drawing of another exemplary embodiment of the process for producing monomers.

FIG. 3 is a schematic drawing of yet another exemplary embodiment of the process for producing monomers.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take a main product from the bottom.

As used herein, the term “rich” can mean an amount of at least generally 50%, and preferably 70%, more preferably 90% or above by mass of a compound or class of compounds in a stream.

As used herein, the term “a component-rich stream” or “a stream rich in a component” means that the rich stream coming out of a vessel has a greater concentration of the component than any other stream from the vessel.

As used herein, the term “a component-lean stream” or “a stream lean in a component” means that the lean stream coming out of a vessel has a smaller concentration of the component than any other stream from the vessel.

As used herein, the term “initial boiling point” (IBP) means the temperature at which the sample begins to boil using a simulated distillation method of ASTM D-7169, ASTM D-86 or D 1160, or TBP, as the case may be.

As used herein, the term “end point” (EP) means the temperature at which the sample has all boiled off using a simulated distillation method of ASTM D-7169, ASTM D-86 or D 1160, or TBP, as the case may be.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx-” refers to molecules with x and preferably x and less carbon atoms. The term “Cx+” refers to molecules with x and preferably x and more carbon atoms.

The term “unit” is to be understood to refer to one or more process steps comprising a chemical transformation. At the heart of a unit is one or more catalytic reactors or separation vessels necessary to accomplish the transformation. A unit may further comprise additional separation vessels including fractionation column(s) to separate product streams. A unit may further comprise pretreatment steps for the chemical transformation. Taken together, “unit” comprises one or more reactors or separation vessels and separation steps and pretreatment steps, whether or not shown in the diagram or explicitly discussed in the specification.

The terms “T10” and “T90” are used here to characterize the volatility of a petroleum fraction. T10 and T90 refer to the temperatures for recovery of 10% and 90%, respectively, in distillation of petroleum products corrected to atmospheric pressure using a laboratory standard method of ASTM D-7169, ASTM D-86 or D 1160, or TBP, as the case may be.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “carbon number” refers to the number of carbon atoms per hydrocarbon molecule.

As used herein, the term “passing” includes “feeding” and means that the material passes from a conduit or vessel to an object.

As used herein, the prefix “bio” as used herein, refers to an association with a renewable resource of biological origin, such resources generally being exclusive of fossil fuels.

As used herein, the term “net” with respect to products means products in the desired boiling range excluding unconverted materials such as unconverted oil.

As used herein, “petroleum stream” or “petroleum feedstock” may refer to crude oil, crude oil refinery distillates, crude oil refinery residue, cracked products or hydrocarbons from a crude oil refinery, liquefied coal, bitumen, typically extracted from the ground or sea floor.

As used herein, the term “vacuum gas oil” (VGO) includes hydrocarbons having an initial boiling point above approximately 343° C. (650° F.), with a T10 boiling point temperature using ASTM D1160 of approximately 370° C. (698° F.) and a T90 boiling point temperature using ASTM D1160 of approximately 500° C. (932° F.).

### DETAILED DESCRIPTION

The present disclosure provides a process for producing monomers. One of the typical routes of plastic circularity includes the conversion of the recycled plastics into a diesel fuel, and burn the resultant diesel fuel which may lead to carbon dioxide emission into the atmosphere. Another route is to process the recycled plastics directly into steam crackers to make olefins or crack plastics into fuels and a minority of olefins within an FCC unit. However, these routes have low carbon efficiency in the conversion to olefins.

The present disclosure provides an efficient process for converting the plastics to monomers. The process provides improved carbon efficiency in the conversion to olefins.

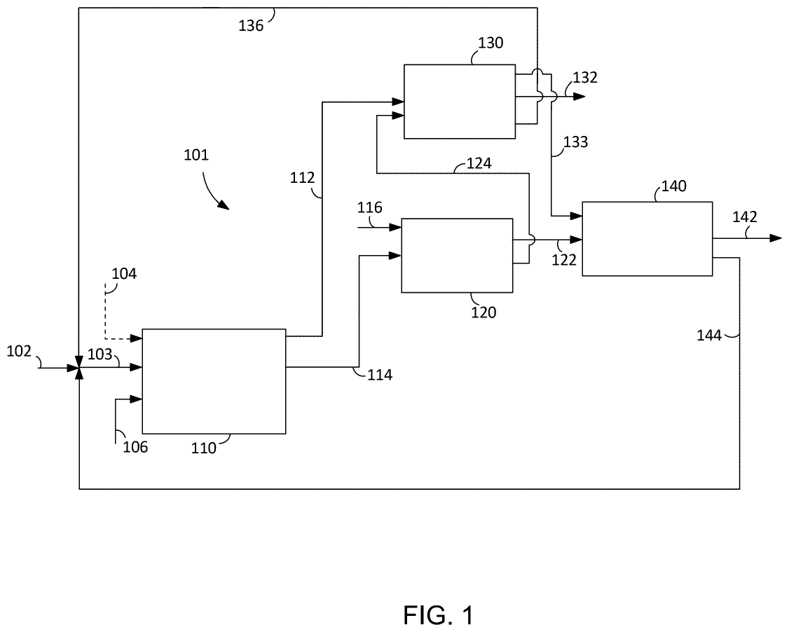

An embodiment of the process for producing monomers 101 is shown in FIG. 1. The process 101 comprises a hydrocracking unit 110, a naphtha to ethane and propane (NEP) unit 120, a propylene producing unit 130, and an ethylene producing unit 140. The feedstock to the process may include a hydrocarbon feed stream in line 102. The hydrocarbon feed stream in line 102 may comprise at least one of a diesel stream, a vacuum gas oil stream, distillate range hydrocarbons, and a pyrolysis oil stream. In an aspect, the hydrocarbon feed stream in line 102 may be produced from a plastic feed stream or a mixed-plastic feed stream or produced from tires such as whole tires and the tires shreds or produced from both plastics and tires. The hydrocarbon feed stream produced from the plastic feed stream may comprise paraffinic and olefinic hydrocarbons. The hydrocarbon feed stream produced from tires may comprise naphthenic and aromatic hydrocarbons. The hydrocarbon feed stream in line 102 may be taken from a pyrolysis reactor which pyrolyzes the plastic feed stream. The hydrocarbon feed stream in line 102 may comprise pyrolysis oil which is entirely produced from a plastic feed stream or tires. The properties of the pyrolysis oil stream produced from both the plastic feed and tires are as shown in Table 1 below:

TABLE 1

| | Pyrolysis | Pyrolysis |
|---|---|---|
| | oil produced | oil produced |
| Property | from plastics | from tires |
| Relative density, 60° F. | 0.77-0.85 | 0.85-1.0 |
| D2887 Distillation, 5 wt % | 60-100 | 100-200 |
| D2887 Distillation, 50 wt % | 150-300 | 200-300 |
| D2887 Distillation, 95 wt % | 350-500 | 300-400 |
| S, wt.-ppm | 50-1000 | 5000-15000 |
| N, wt.-ppm | 100-2500 | 4000-7000 |
| Br No. | 50-100 | 50-100 |
| Diene Index | 1-10 | 1-10 |
| Oxygen, wt.-% | 0.1-1.0 | 0.5-2.0 |
| Total Acid Number | 1-5 | 1-5 |
| Chloride, wt.-ppm | 100-500 | 10-100 |

The hydrocarbon feed stream in line 102 may be taken from the pyrolysis reactor and charged to the hydrocracking unit 110. Optionally, a fossil feed such as a petroleum stream in line 104 may be charged to the hydrocracking unit 110. In an embodiment, the hydrocarbon feed stream in line 102 may comprise a fossil feed such as a petroleum stream and from about 1 to about 99 wt % a pyrolysis oil stream produced from plastics or tires or both. A hydrocracking hydrogen stream in line 106 is also passed to the hydrocracking unit 110. In the hydrocracking unit 110, the hydrocarbon feed stream in line 102 and optionally the petroleum stream in line 104, may be hydrocracked over a hydrocracking catalyst in the presence of the hydrocracking hydrogen stream at hydrocracking conditions to open rings present in the pyrolysis oil stream to produce a net hydrocracked effluent stream.

The hydrocracking unit 110 may be operated in several different non-limiting hydrocracking configurations such as a once-through, single stage recycle and two-stage hydrocracking configurations. In an embodiment, the hydrocracking unit 110 may comprise a single stage hydrocracking reactor. The hydrocarbon feed stream in line 102 and the hydrocracking hydrogen stream are charged to the hydrocracking reactor. The hydrocarbon feed stream in line 102 is hydrocracked over a hydrocracking catalyst in the hydrocracking reactor to produce a net hydrocracked effluent stream. The hydrocarbon feed stream in line 102 may be hydrotreated by contact with a pre-hydrotreating catalyst in the hydrocracking unit 110. A hydrotreated pyrolysis oil stream may be charged to the hydrocracking reactor. Hydrotreating is a process wherein hydrogen is contacted with hydrocarbon in the presence of hydrotreating catalysts which are primarily active for the removal of heteroatoms, such as sulfur, nitrogen, chlorine, and metals from the hydrocarbon feedstock. In hydrotreating, olefinic hydrocarbons with double and triple bonds may be saturated.

Suitable hydrotreating catalysts for use in the hydrocracking unit 110 may include any known conventional hydrotreating catalysts and include those which are comprised of at least one Group VIII metal, preferably iron, cobalt and nickel, more preferably cobalt and/or nickel and at least one Group VI metal, preferably molybdenum and tungsten, on a high surface area support material, preferably alumina. Other suitable hydrotreating catalysts include zeolitic catalysts. In the high sulfur and nitrogen environment of the hydrocracking unit 110, noble metal catalysts would be discouraged. More than one type of hydrotreating catalyst may be used in the hydrocracking unit 110. The Group VIII metal is typically present in an amount ranging from about 2 to about 20 wt %, preferably from about 4 to about 12 wt %. The Group VI metal will typically be present in an amount ranging from about 1 to about 25 wt %, preferably from about 2 to about 25 wt %.

Preferred reaction conditions for hydrotreating may include a temperature from about 290° C. (550° F.) to about 455° C. (850° F.), suitably 316° C. (600° F.) to about 427° C. (800° F.) and preferably 343° C. (650° F.) to about 399° C. (750° F.), a pressure from about 2.1 MPa (gauge) (300 psig), preferably 4.1 MPa (gauge) (600 psig) to about 20.6 MPa (gauge) (3000 psig), suitably 13.8 MPa (gauge) (2000 psig), preferably 12.4 MPa (gauge) (1800 psig), a liquid hourly space velocity of the fresh hydrocarbon feed stream from about 0.1 hr⁻, suitably 0.5 hr⁻¹, to about 10 hr⁻¹, preferably from about 1.5 to about 8.5 hr⁻¹, and a hydrogen rate of about 168 Nm3/m3 (1,000 scf/bbl), to about 1,011 Nm3/m3 oil (6,000 scf/bbl), preferably about 168 Nm3/m3 oil (1,000 scf/bbl) to about 674 Nm3/m3 oil (4,000 scf/bbl), with a hydrotreating catalyst or a combination of hydrotreating catalysts.

The hydrotreated pyrolysis oil stream may be hydrocracked in the hydrocracking reactor. Hydrocracking is a process in which hydrocarbons crack in the presence of hydrogen to lower molecular weight hydrocarbons. The hydrocracking reactor may be a fixed bed reactor that comprises one or more vessels, single or multiple catalyst beds in each vessel, and various combinations of hydrotreating catalyst, hydroisomerization catalyst and/or hydrocracking catalyst in one or more vessels. The hydrocracking reactor may be operated in a conventional continuous gas phase, a moving bed or a fluidized bed hydrocracking reactor.

The hydrocracking catalyst may utilize cracking bases comprising amorphous silica-alumina or zeolites combined with one or more Group VIII or Group VIB metal hydrogenating components if mild hydrocracking is desired to produce a balance of middle distillate and gasoline. In another aspect, when light naphtha and LPG, which are aliphatic hydrocarbons or six carbon numbers and lower, are significantly preferred in the converted product over gasoline or distillate production, partial or complete hydrocracking conversion to aliphatic hydrocarbons of six carbon numbers or less may be performed in the hydrocracking reactor with a catalyst which comprises, in general, any crystalline zeolite cracking base upon which is deposited a Group VIII metal hydrogenating component. Additional hydrogenating components may be selected from Group VIB for incorporation with the zeolite base.

The zeolites in hydrocracking bases are sometimes referred to in the art as molecular sieves and are usually composed of silica, alumina and one or more exchangeable cations such as sodium, magnesium, calcium, rare earth metals, etc. They are further characterized by crystal pores of relatively uniform diameter between about 4 and about 14 Angstroms. It is preferred to employ zeolites having a relatively high silica/alumina mole ratio between about 3 and about 12. Suitable zeolites found in nature include, for example, mordenite, stilbite, heulandite, ferrierite, dachiardite, chabazite, erionite and faujasite. Suitable synthetic zeolites include, for example, the B, X, Y and L crystal types, e.g., synthetic faujasite and mordenite. The preferred zeolites are those having crystal pore diameters between about 8 and 12 angstroms, wherein the silica/alumina mole ratio is about 4 to 6. One example of a zeolite falling in the preferred group is synthetic Y molecular sieve.

The natural occurring zeolites are normally found in a sodium form, an alkaline earth metal form, or mixed forms. The synthetic zeolites are nearly always prepared first in the sodium form. In any case, for use as a cracking base it is preferred that most or all of the original zeolitic monovalent metals be ion-exchanged with a polyvalent metal and/or with an ammonium salt followed by heating to decompose the ammonium ions associated with the zeolite, leaving in their place hydrogen ions and/or exchange sites which have actually been decationized by further removal of water. Hydrogen or “decationized” Y zeolites of this nature are more particularly described in U.S. Pat. No. 3,100,006.

Mixed polyvalent metal-hydrogen zeolites may be prepared by ion-exchanging first with an ammonium salt, then partially back exchanging with a polyvalent metal salt and then calcining. In some cases, as in the case of synthetic mordenite, the hydrogen forms can be prepared by direct acid treatment of the alkali metal zeolites. In one aspect, the preferred cracking bases are those which are at least about 10 wt %, and preferably at least about 20 wt %, metal-cation-deficient, based on the initial ion-exchange capacity. In another aspect, a desirable and stable class of zeolites is one wherein at least about 20 wt % of the ion exchange capacity is satisfied by hydrogen ions.

The active metals employed in the preferred hydrocracking catalysts of the present invention as hydrogenation components are those of Group VIII, i.e., iron, cobalt, nickel, ruthenium, rhodium, palladium, osmium, iridium and platinum. In another embodiment, Group VIII metals such as nickel or cobalt may be used to promote the aromatic saturation and intermediates re-hydrogenation activity of Group VIB metals. Such Group VIB hydrogenation metals may comprise molybdenum and tungsten. The amount of hydrogenating metal in the catalyst can vary within wide ranges. Broadly speaking, any amount between about 0.05 wt % and about 30 wt % may be used. In the case of noble metals, it is normally preferred to use about 0.05 to about 2 wt % noble metal.

The method for incorporating the hydrogenation metal is to contact the base material with an aqueous solution of a suitable compound of the desired metal. Following addition of the selected hydrogenation metal or metals, the resulting catalyst powder is then filtered, dried, pelleted with added lubricants, binders or the like if desired, and calcined in air at temperatures of, e.g., about 371° C. (700° F.) to about 648° C. (1200° F.) in order to activate the catalyst and decompose ammonium ions. Alternatively, the base component may first be pelleted, followed by the addition of the hydrogenation metal and activation by calcining. In yet another embodiment the base component may first be pelleted, followed by the addition of the hydrogenation metal and dried.

The foregoing catalysts may be employed in undiluted form, or the powdered catalyst may be mixed and copelleted with other relatively less active catalysts, diluents or binders such as alumina, silica gel, silica-alumina cogels, activated clays and the like in proportions ranging between about 5 and about 90 wt %. These diluents may be employed as such or they may contain a minor proportion of an added hydrogenating metal such as a Group VIB and/or Group VIII metal. Additional metal promoted hydrocracking catalysts may also be utilized in the process of the present invention which comprises, for example, aluminophosphate molecular sieves, crystalline chromosilicates and other crystalline silicates. Crystalline chromosilicates are more fully described in U.S. Pat. No. 4,363,718.

In an embodiment, the hydrocracking conditions in the hydrocracking reactor may include a temperature from about 290° C. (550° F.) to about 468° C. (875° F.), preferably 343° C. (650° F.) to about 445° C. (833° F.), a pressure from about 4.8 MPa (gauge) (700 psig) to about 20.7 MPa (gauge) (3000 psig), a liquid hourly space velocity (LHSV) of about 0.3 to about 1.5 hr⁻¹, suitably no more than about 1.0 hr⁻ and preferably about 0.4 to about 0.7 hr⁻ and a hydrogen rate of about 421 Nm3/m3 (2,500 scf/bbl) to about 2,527 Nm3/m3 oil (15,000 scf/bbl). Hydrogen partial pressure may be 1 MPa (abs) (1500 psia) to about 1.7 MPa (abs) (2500 psia).

The hydrocracked effluent stream may be separated in a separator to provide a hydrocarbonaceous, hot gaseous stream and a hydrocarbonaceous, hot liquid stream. A hydrogen rich gas stream may be separated from the hot gaseous stream and recycled to the hydrocracking reactor. The hot liquid stream may be stripped in a stripping column with a stripping media which is an inert gas such as steam to provide a stripper gaseous stream of naphtha, hydrogen, hydrogen sulfide, steam and other gases in a stripper overhead stream. is fed to a fractionation column. The stripper gaseous stream may be condensed to provide a net stripper overhead stream comprising C₃₋ hydrocarbons. Some C₄ may be present in the net stripper overhead stream.

The stripping column may be operated with a bottoms temperature between about 149° C. (300° F.) and about 360° C. (680° F.) or about 160° C. (320° F.) to about 288° C. (550° F.), and an overhead pressure of about 0.35 MPa (gauge) (50 psig), preferably no less than about 0.50 MPa (gauge) (72 psig), to no more than about 2.0 MPa (gauge) (290 psig).

A stripped stream may be taken from the bottoms of the stripping column and fed to a fractionation column. The product streams from the fractionation column may include a net overhead stream comprising liquefied petroleum gas and a naphtha stream. In an aspect, the net overhead stream may comprise a light naphtha stream and a bottoms stream comprising heavy naphtha may be discharged from the fractionation column.

In an embodiment, the hydrocracking unit 110 may comprise a two-stage hydrocracking reactor. The hydrocarbon feed stream in line 102 may be hydrocracked in a first hydrocracking reactor. The first hydrocracking reactor may be operated at the operating conditions as earlier described. The first hydrocracking reactor may comprise one or more hydrocracking catalyst as earlier described. The hydrocracked effluent stream may be fractionated in the fractionation column. A net fractionation bottoms stream may be recycled as a recycle oil (RO) stream to the second hydrocracking reactor. The second hydrocracking reactor may comprise the same as or different hydrocracking catalyst than the first hydrocracking reactor or may have some of the same as and some different than the first hydrocracking reactor. The second hydrocracking reactor may be operated at similar operating conditions as earlier described. The second hydrocracking reactor may comprise one or more hydrocracking catalysts as earlier described. The second hydrocracking reactor saturates remaining aromatics to naphthenes and hydrocracks naphthenes to aliphatics in the second hydrocracking reactor.

A net hydrocracked effluent stream may exit the second hydrocracking reactor and be combined with the hydrocracked effluent stream from the first hydrocracking reactor. A combined hydrocracked effluent stream may be separated and fractionated as previously described.

As shown in FIG. 1, a naphtha stream is discharged in line 114 from the hydrocracking unit 110. The naphtha stream in line 114 may comprise C₄ to C₇ hydrocarbons. In an aspect, the naphtha stream in line 114 may comprise C₄ to C₆ hydrocarbons, preferably C₄ to C₆ aliphatic hydrocarbons. A C₃₋ hydrocarbon stream is discharged in line 112 from the hydrocracking unit 110. The naphtha stream in line 114 may be fed to the NEP unit 120. A hydrogen stream in line 116 is passed to the NEP unit 120. In the NEP unit 120, the naphtha stream in line 114 is contacted with a NEP catalyst and hydrogen to produce a light paraffinic stream. The NEP unit 120 may comprise a NEP reactor, and an NEP separation unit. The naphtha stream in line 114 may be combined with the hydrogen stream in line 116 to provide a charge stream and charged to the NEP reactor to be contacted with an NEP catalyst. The naphtha stream may be heated to a reaction temperature of about 300° C. to about 600° C., suitably between about 325° C. and about 550° C., and preferably between about 350° C. and about 525° C. in the NEP reactor. A total pressure in the NEP reactor should be about 0.1 to about 3 MPa (abs), preferably greater than 1 MPa (abs).

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙA1ₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures of thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce 1/888-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steam oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

After paraffin conversion, a light paraffinic stream is discharged from the NEP reactor. The light paraffinic stream may comprise at least about 40 wt % ethane or at least about 40 wt % propane or at least about 70 wt % and preferably at least about 80 wt % ethane and propane. The light paraffinic stream may be cooled and fed to an NEP separation unit. The NEP separation unit may be a fractionation column or a series of fractionation columns and other separation units. The NEP separation unit may comprise a demethanizer column that separates the light paraffinic stream into a gas stream in an overhead line and a C₂₊ paraffin stream in a bottoms line. The gas stream may be sent to a hydrogen purification unit such as a PSA unit to recover hydrogen for recycle to the NEP reactor. Remaining methane from the hydrogen purification unit may be used for fuel gas. The C₂₊ paraffin stream may then be fed to a deethanizer column to produce the ethane stream in a deethanizer overhead line 132 and a C₃+ paraffin stream in a deethanized bottoms line. The C₃₊ paraffin stream may then be fed to a depropanizer column to produce the propane stream in a depropanizer overhead line and the heavy paraffin stream which may comprise C₄₊ hydrocarbons. The NEP separation unit may take other forms. The C₄₊ hydrocarbons stream may be provided in line 136 from the propylene producing unit 130. The C₄₊ hydrocarbons stream in line 136 is primarily a mixed C₄ stream. In an embodiment, the C₄₊ hydrocarbons stream in line 136 may be recycled to the hydrocracking unit 110. In an exemplary embodiment, the C₄₊ hydrocarbons stream in line 136 may be combined with the hydrocarbon feed stream in line 102 and charged to the hydrocracking unit 110 in a charge line 103. In an aspect, the C₄₊ hydrocarbons stream in line 136 may be treated before recycling to the hydrocracking unit 110. A portion or an entirety of the C₄₊ hydrocarbons stream in line 136 may be recycled to the hydrocracking unit 110. Recycling the C₄₊ hydrocarbons stream in line 136 to the hydrocracking unit 110 can increase the net olefin product from the same amount of feed.

An ethane stream is discharged in line 122 from the NEP unit 120. The ethane stream in line 122 may be charged to the ethylene producing unit 140 in which ethane in the ethane stream is converted into ethylene. In an aspect, an ethane containing stream in line 133 may be taken from the propylene producing unit 130 and fed to the ethylene producing unit 140 for producing ethylene. The ethane containing stream in line 133 may comprise ethane that may be present in the feed to the propylene producing unit 130 which includes a propane stream in line 124 and the C₃₋ hydrocarbon stream in line 112. Further, the ethane containing stream in line 133 may comprise ethane that may be produced in the propylene producing unit 130. In an embodiment, the ethylene producing unit 140 is a steam cracking unit. The ethane stream in line 122 may be cracked under steam in a furnace to produce a cracked stream including an ethylene stream. The ethane stream may be charged to the ethane steam cracking unit in the gas phase. The ethane steam cracking unit may preferably be operated at a temperature of about 750° C. (1382° F.) to about 950° C. (1742° F.). An ethylene stream is discharged in line 142 from the steam cracking unit 140.

In an embodiment, a C₃₊ hydrocarbons comprising propane, butane, butadiene, butene, mixed C₅, and C₆₊ components are taken in a collective C₃₊ hydrocarbon stream in line 144 and recycled to the NEP unit 120. In an exemplary embodiment, the C₃₊ hydrocarbons stream in line 144 may be combined with the hydrocarbon feed stream in line 102 and charged to the hydrocracking unit 110 in a charge line 103. In an aspect, a portion or an entirety of the C₃₊ hydrocarbons stream in line 144 may be recycled to the hydrocracking unit 110. A primary advantage of recycling the C₃₊ hydrocarbons stream in line 144 with the hydrocarbon feed stream in line 102 to the hydrocracking unit 110 is that the combined stream can be utilized without separation or additional treatment. Also, recycling the C₃₊ hydrocarbons stream in line 144 to the hydrocracking unit 110 can increase the net olefin product from the same amount of feed.

A propane stream is discharged in line 124 from the NEP unit 120. The propane stream in line 124 may be charged to a propylene producing unit 130 in which propane in the propane stream is converted into propylene. In an embodiment, the propylene producing unit 130 may be a propane dehydrogenation (PDH) unit. The C₃₋ hydrocarbon stream in line 112 may be charged to the PDH unit 130 to convert C₃ into propylene. PDH catalyst is used in a dehydrogenation reaction process to catalyze the dehydrogenation of propane. The conditions in the dehydrogenation reactor may include a temperature of about 500 to about 800° C., a pressure of about 40 to about 310 kPa (abs) and a catalyst to oil ratio of about 5 to about 100.

The dehydrogenation reaction may be conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated in a regenerator. The regenerator may combust coke from the dehydrogenation catalyst and fuel gas to ensure sufficient enthalpy in the dehydrogenation reactor to promote the endothermic reaction.

The dehydrogenation catalyst selected should minimize cracking reactions and favor dehydrogenation reactions. Suitable catalysts for use herein include an active metal which may be dispersed in a porous inorganic carrier material such as silica, alumina, silica alumina, zirconia, or clay. An exemplary embodiment of a catalyst includes alumina or silica-alumina containing gallium, a noble metal, and an alkali or alkaline earth metal.

The catalyst support comprises a carrier material, a binder and an optional filler material to provide physical strength and integrity. The carrier material may include alumina or silica-alumina. Silica sol or alumina sol may be used as the binder. The alumina or silica-alumina generally contains alumina of gamma, theta and/or delta phases. The catalyst support particles may have a nominal diameter of about 400 to about 5000 micrometers with the average diameter of about 600 to about 3500 micrometers. Preferably, the surface area of the catalyst support is about 85 to about 140 m²/g.

The fluidized dehydrogenation catalyst may comprise a dehydrogenation metal on a support. The dehydrogenation metal may be a one or a combination of transition metals. A noble metal may be a preferred dehydrogenation metal such as platinum or palladium. Gallium is an effective metal for paraffin dehydrogenation. Metals may be deposited on the catalyst support by impregnation or other suitable methods or included in the carrier material or binder during catalyst preparation.

The acid function of the catalyst should be minimized to prevent cracking and favor dehydrogenation. Alkali metals and alkaline earth metals may also be included in the catalyst to attenuate the acidity of the catalyst. Rare earth metals may be included in the catalyst to control the activity of the catalyst. Concentrations of 0.001% to 10 wt % metals may be incorporated into the dehydrogenation catalyst. In the case of the noble metals, it is preferred to use about 10 parts per million (ppm) by weight to about 600 ppm by weight noble metal. More preferably it is preferred to use about 10 to about 100 ppm by weight noble metal. The preferred noble metal is platinum. Gallium should be present in the range of 0.3 wt % to about 3 wt %, preferably about 0.5 wt % to about 2 wt %. Alkali and alkaline earth metals may be present in the range of about 0.05 wt % to about 1 wt %.

Regenerated catalyst may be contacted with the propane in line 124 and in line 112 perhaps with a fluidizing gas to lift the propane stream and dehydrogenation catalyst up a riser while dehydrogenation occurs. Above the riser spent dehydrogenation catalyst and propylene product may be separated by a centripetal separation device. Propylene product gas may be quenched with a cooling fluid to prevent over reaction to undesired by-products. Separation of the propylene product from the PDH effluent stream may include quench contacting and fractionation to produce a propylene product stream. Unreacted propane may be recycled to the dehydrogenation reactor and lighter gases may be recycled to the regenerator as fuel gas to be combusted to provide enthalpy for the reaction.

FIG. 1 shows charging the C₃₋ hydrocarbon stream in line 112 from the hydrocracking unit 110 into the propylene producing unit 130. In an alternate embodiment, at least a portion of the C₃₋ hydrocarbon stream in line 112 may be charged to the NEP reactor and passed to the downstream propylene producing unit 130 therefrom.

The propylene producing unit 130 may also employ a catalytic moving bed reactor. The reactor section may comprise several radial flow reactors in parallel or series heated by charge and interstage heaters. The propane stream perhaps with added hydrogen flows in each dehydrogenation reactor from a screened center pipe through an annular dehydrogenation catalyst bed to an outer effluent annulus. Flow may be in the reverse fashion. The dehydrogenation catalyst may comprise a noble metal or mixtures thereof, a modifier selected from the group consisting of alkali metals or alkaline-earth metals and mixtures thereof, a component selected from the group consisting of tin, germanium, lead, indium, gallium, thallium, and mixtures thereof, and a porous support forming a catalyst particle. The catalyst support may comprise oil dropped alumina spheres.

Dehydrogenation conditions may include a temperature of from about 400 to about 900° C., a pressure of from about 0.01 to 10 atmospheres absolute, and a liquid hourly space velocity (LHSV) of from about 0.1 to 100 hr-1. In an embodiment, the dehydrogenation conditions may include a reactor inlet temperature of from about 580 to about 640° C. and a reactor outlet pressure of about atmospheric pressure. The pressure in the dehydrogenation reactor is maintained as low as practicable, consistent with equipment limitations, to maximize chemical equilibrium advantages. Spent dehydrogenation catalyst in the annular catalyst bed may be withdrawn from the bottom of the bed, forwarded to a regenerator to combust coke from the catalyst with air at about 450 to about 600° C. Noble metal on the catalyst may be redispersed by an oxyhalogenation process, dried and returned to the top of the dehydrogenation catalyst bed as regenerated dehydrogenation catalyst.

Dehydrogenation effluent may be cooled, compressed, dried and hydrogen is cryogenically separated from the hydrocarbons with a net gas purity of 85 to 93 mol % hydrogen. Hydrocarbon liquid is selectively hydrogenated to convert diolefins and acetylenes and the hydrocarbon liquid is fractionated in a deethanizer column to remove ethane and propylene is split from propane in a propane-propylene splitter column to provide polymer-grade propylene. Propane may be recycled as feed to the PDH unit 130. A propylene stream is discharged in line 132 from the PDH unit 130.

The ethylene stream in line 142 and the propylene stream in line 132 may be polymerized to produce polyolefins, polyethylene and polypropylene respectively. The ethylene and propylene may be purified to produce polymer-grade ethylene and the propylene before polymerization. The ethylene stream in line 142 and the propylene stream in line 132 may be separately passed through cryogenic distillation to produce polymer-grade ethylene and the propylene monomers. In some aspect, the ethylene stream in line 142 and the propylene stream in line 132 may be separately passed through a selective hydrogenation step over catalysts to remove propane, ethane and/or trace amounts of acetylene to produce polymer-grade ethylene and the propylene monomers.

Polyolefins such as polyethylene and polypropylene may be prepared by a gas phase or slurry polymerization process, which involves the polymerization of olefin monomer with the aid of catalyst and optionally, if required, depending on the used catalyst, a co-catalyst. Following such polymerization process, a polymerization effluent is produced comprising polymer solids in a fluid that contains unreacted monomer, and optionally unreacted comonomer.

The polymer-grade ethylene may be polymerized to produce ethylene polymers. One type of ethylene polymer is polyethylene. Ethylene polymers may, for example, be produced by catalytic polymerization processes or alternatively via free-radical high-pressure polymerization processes. Catalytic polymerization processes, for example, include processes in which catalyst systems of the Ziegler type, of the Phillips type, and/or of the single-site type are used.

Propylene polymers or polypropylenes are suitable for many applications. For instance polypropylene (PP) is applicable in areas where sealing properties play an important role, like in the food or medical packing industry. Propylene polymers may be produced by catalytic polymerization processes by contacting the polymer-grade propylene with a catalyst under polymerization conditions. One or more types of catalyst may be used in one or more reactors to produce polypropylene.

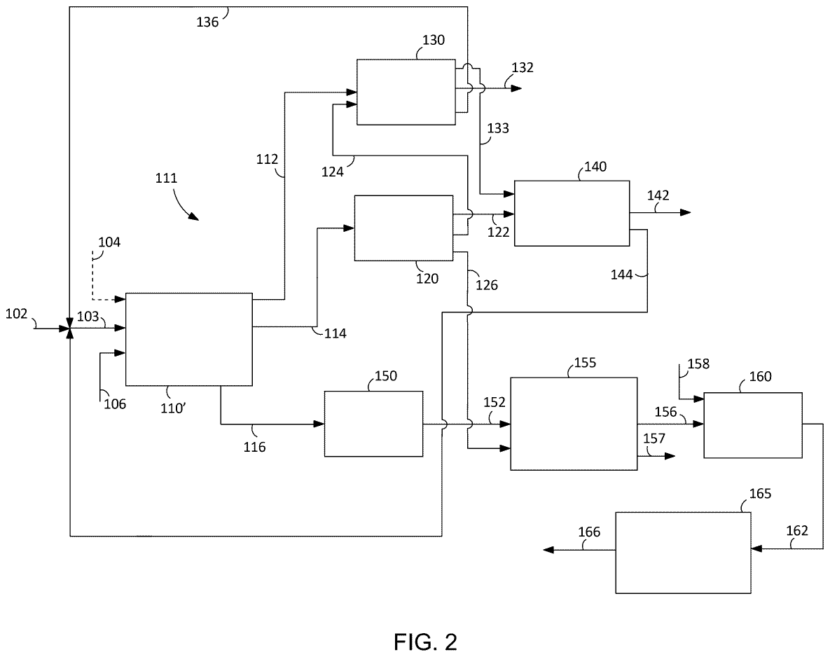

Another exemplary embodiment of the process for producing monomers 111 is shown in FIG. 2. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). ‘The configuration and operation of the embodiment of FIG. 2 is essentially the same as in FIG. 1 with the following exceptions.

In the embodiment shown in FIG. 2, the hydrocracking unit 110′ comprises producing a light naphtha stream and a heavy naphtha stream. The C₃₋ hydrocarbon stream may be taken from the stripping column of the hydrocracking unit 110′. The light naphtha stream may be taken from the overhead of the fractionation column, and the heavy naphtha stream, may be taken from the bottom of the fractionation column. The light naphtha stream is discharged in line 114 from the hydrocracking unit 110′. In an aspect, the light naphtha stream in line 114 may comprise C₄ to C₆ hydrocarbons, preferably C₄ to C₆ aliphatic hydrocarbons. The light naphtha stream in line 114 is charged to the NEP unit and processed as earlier described in FIG. 1. The C₃₋ hydrocarbon stream in line 112 is charged to the dehydrogenation unit 130 and processed as earlier described in FIG. 1.

As shown in FIG. 2, the process 111 additionally comprises a reforming unit 150, an aromatics complex 155, an oxidation unit 160, and a PET melt unit 165. The heavy naphtha stream is discharged in line 116 from the hydrocracking unit 110′. The heavy naphtha stream in line 116 may comprise C₆₊ hydrocarbons, preferably C₇-C₁₀ hydrocarbons. In an aspect, the heavy naphtha stream in line 116 may comprise C₆-C₁₂ paraffins, preferably C₆-C₉ paraffins, or more preferably C₆-C₈ paraffins. In an embodiment, the heavy naphtha stream in line 116 is charged to the reforming unit 150. The reforming unit 150 may comprise a reformer reactor in which the heavy naphtha stream in line 116 is contacted with a reforming catalyst to produce an aromatics stream.

In the reformer reactor, the heavy naphtha stream in line 116 may contact the catalyst in individual reactors in either upflow, downflow, or radial flow fashion, with the radial flow mode being preferred. The catalyst is contained in a fixed-bed system or a moving-bed system with associated continuous catalyst regeneration. The reformer reactor may be operated at a temperature of from about 400° C. to about 6000° C., or from about 450° C. to about 560° C. The reformer reactor may be operated at a pressure of about 413 kPa (gauge) (60 psig) to about 2758 kPa (gauge) (400 psig) or a lower pressure. The reformer reactor converts the paraffinic and naphthenic materials to aromatics to produce a reformed stream comprising C₁ to C₅ hydrocarbons, C₆₊ aromatic compounds such as C₆, C₇, and heavy aromatics such as C₈+, and hydrogen. In an aspect, the reformed stream may comprise a higher concentration of C₆-C₁₁ aromatics than the heavy naphtha stream in line 116.

Suitable catalysts for use in the reformer reactor may include a dual-function catalyst having a multi-metallic, combination of two or more metal components in specified concentrations on the finished catalyst, and its use in hydrocarbon conversion with increased aromatics production. Catalysts having both a hydrogenation-dehydrogenation function and a cracking function should maximize the former and minimize the latter. The cracking function generally relates to an acid-action material of the porous, adsorptive, refractory-oxide type which is typically utilized as the support or carrier for a heavy-metal component, such as the Group VIII (IUPAC 8-10) metals, which primarily contribute the hydrogenation-dehydrogenation function. Other metals in combined or elemental form can influence one or both of the cracking and hydrogenation-dehydrogenation functions.

Catalytic reforming involves a number of competing processes or reaction sequences. These include dehydrogenation of cyclohexanes to aromatics, dehydroisomerization of alkylcyclopentanes to aromatics, dehydrocyclization of an acyclic hydrocarbon to aromatics, dealkylation of alkylbenzenes and isomerization of paraffins. Hydrocracking reactions which produce light paraffin gases have a deleterious effect on the yield of products boiling in the jet fuel range. Process improvements in catalytic reforming thus are targeted toward enhancing those reactions effecting a higher yield of the liquid products containing more 5 or more carbon atoms and minimizing those reactions affecting cracked products containing 4 or fewer carbon atoms.

Generally, it is desirable to have flexibility with catalyst functionality for reforming. In one exemplary reforming process, increasing the yield of one or more C₅₊ hydrocarbons, hydrogen, and aromatic yields is desired. Optionally, the acidity of the catalyst can be altered by adding a metal and/or other elements to the catalyst. Generally, modification of the acid function results in reduced cracking of the alkanes to C₃ and C₄ light ends allowing increased selectivity to the formation of aromatics. Modification of the metal function may also occur resulting in the reduction of alkane cracking to methane and ethane. There can also be a reduction in the dealkylation reactions of aromatics leaving heavier and more valuable C₈₊ aromatics.

Beside the yields, the activity of a catalyst may enable obtaining a commercially useful conversion level without employing additional quantities of catalyst or using excessively high temperatures, which can lead to undesired higher costs. Higher catalyst activity can also be utilized to process greater quantities of feed or to increase conversion, and therefore increase the production of valuable products.

Catalytic materials used for reforming paraffinic feeds more selectively towards aromatics can be achieved by tuning the material acidity. In one embodiment, the catalytic material comprises a refractory aluminum oxide support, a metal from the platinum group, such as Re, and a halogen element.

In an embodiment, the catalysts for use in the reformer reactor may comprise a zeolite based catalyst with greater than up to about 1 wt % of a noble metal, or a chlorinated alumina based catalyst with greater than 0 up to about 1 wt % of a noble metal, and one or more metals from tin, germanium, gallium, indium, and rhenium, or combinations thereof.

A reformed stream comprising an increased concentration of aromatics than the heavy naphtha stream leaves the reforming unit in line 152. In an aspect, the reformed stream in line 152 may comprise greater than about 50 wt %, suitably greater than 70 wt % or preferably greater than about 90 wt %, of aromatic hydrocarbons.

In an embodiment, the reformed stream in line 152 may be charged to the aromatics complex 155 to produce xylene. In an embodiment, an aromatics stream in line 126 may be provided from the NEP unit 120 and fed to the aromatics complex 155. The aromatics stream in line 126 may be an aromatics product stream produced from the NEP unit 120. This aromatics stream in line 126 primarily comprises benzene, toluene, ethylbenzene, and xylene (BTEX) and heavier aromatics, such as, A9+ aromatics. For example, the aromatics stream in line 126 comprises 80 wt % or more of BTEX, with the remainder being heavier A9+ aromatics. Feeding the aromatics stream in line 126 to the aromatics complex 155 can increase the virgin plastics production rate from a constant feed. The aromatics complex 155 may comprise at least one of an aromatic extraction unit, a clay treater, a BTX fractionation zone, a transalkylation unit, a xylene fractionation zone, a xylene extraction unit, a heavy aromatics column, and an isomerization unit.

The reformed stream in line 152 may be passed to the clay treater for the removal of any alkylates and olefins that may be present in the reformed stream. A treated reformed stream may be passed to the xylene fractionation zone. The xylene fractionation zone may comprise a xylene column operated at conditions suitable for forming an overhead xylene containing stream. The xylene containing stream may comprise more than about 98 wt % mixed xylenes. The xylene fractionation zone also produces a heavy bottom stream comprising C₉₊ hydrocarbons. The heavy bottom stream may be fed to the heavy aromatics column to provide a heavy aromatics overhead stream comprising C₉ and C₁₀ aromatics that is fed to the transalkylation unit. The heavy aromatics column also produces a heavy aromatics bottom stream comprising C₁₁₊ hydrocarbons. The heavy aromatics column may be operated at a pressure of about 5 kPa (0.7 psia) to about 1,800 kPa (260 psia) and a temperature of about 100° C. (212° F.) to about 360° C. (680° F.).

The xylene containing stream may be introduced into a xylene extraction unit that extracts a selected xylene isomer from non-selected xylene isomers that comprises a xylene raffinate stream. The xylene extraction unit may be based on an adsorption process or a crystallization process or any combination of both. In an aspect, the xylene extraction unit may include a selective adsorbent that preferentially sorbs the selected xylene isomer relative to the other xylene isomers. The xylene raffinate stream may be fed to an isomerization unit to isomerize the non-selected xylene isomers to produce more of the selected xylene isomer. In an embodiment, the selected xylene isomer is paraxylene. The isomerized effluent from the isomerization unit may be recycled to the xylene fractionation zone. The selected paraxylene isomer stream exits the xylene extraction unit and discharged from the aromatic complex 155 in selected xylene line 156. A heavy aromatics stream may be taken in line 157 from the aromatics complex 155. The heavy aromatics stream in line 157 may be recycled to the hydrocracking unit 110.

In an aspect, the paraxylene stream in line 156 may be charged to the PTA production unit to produce purified terephthalic acid. Terephthalic acid is an important compound with a variety of applications. The primary use of terephthalic acid is as a feedstock in the production of polyethylene terephthalate (PET). PET is a well-known plastic used in great quantities around the world to make products such as bottles, fibers, and packaging.

In an embodiment, the oxidation unit 160 is a liquid-phase oxidation unit. An oxidant stream in line 158 is passed to the liquid-phase oxidation unit 160. In the liquid-phase oxidation unit 160, the liquid-phase paraxylene stream is contacted with a gas-phase oxidant stream which contains molecular oxygen in a liquid-phase oxidation reactor. At least a portion of the molecular oxygen introduced into the reactor as a gas dissolves into the liquid phase of the reaction medium to provide oxygen availability for the liquid-phase reaction. In the liquid-phase oxidation reactor, the molecular oxygen gets dissolved into the liquid phase paraxylene. After the dissolution, a crude terephthalic acid leaves the liquid-phase oxidation reactor. The crude terephthalic acid may be further oxidized in a secondary oxidation reactor to form purer terephthalic acid. A purified terephthalic acid is taken out from the secondary oxidation reactor. A purified terephthalic acid stream may be discharged in product line 162 from the liquid-phase oxidation unit 160.

Further, the liquid-phase oxidation unit 160 may produce dimethyl terephthalate (DMT) from liquid-phase paraxylene stream. The oxidant stream in line 158 oxidizes p-xylene with molecular oxygen under liquid phase conditions in the presence of acetic acid and a catalyst system consisting of one or more heavy metals and a side-chain oxidation initiator or promoter such as a source of bromine or acetaldehyde or a methylenic ketone (a ketone having one methyl group and one hydrocarbon group other than a methyl group attached to the carbonyl carbon). Such a catalytic oxidation process may be conducted by batchwise or semi-continuous or continuous operation and produces a fluid reaction effluent which is depressurized and cooled to crystallize substantially all of the terephthalic acid as impure product. The impure terephthalic acid is then recovered by some solid-liquid separation, such as filtration, centrifugation, decantation and the like, dried with or without a prior wash to remove adhering mother liquor. Dry terephthalic acid combined with an excess of methanol, such as 3 to 50 parts of methanol per part of terephthalic acid by weight, are reacted with or without an esterification catalyst at a temperature above 150° C. and under pressure to maintain a liquid phase of methanol. The esterification produces a liquid effluent containing DMT together with by-product water, some monoester and other impurities dissolved in the excess methanol. Impure DMT is recovered from the liquid esterification effluent by any one of the several known crystallization techniques and filtration of the resulting slurry of crystalline DMT. The crystalline DMT may be discharged in product line 162 from the liquid-phase oxidation unit 160.

The purified terephthalic acid may be polymerized to produce polyethylene terephthalate (PET). Polyethylene terephthalate (PET) is the most widely used polyester class and is characterized by high modulus, low shrinkage, heat set stability, light fastness and chemical resistance account for the great versatility of PET. A product stream in line 162 comprising at least one of the purified terephthalic acid stream and the DMT may be taken from the liquid-phase oxidation unit 160 and charged to the PET melt unit 165. In the PET melt unit 165, the product stream comprising at least one of the purified terephthalic acid stream and the DMT may be polymerized to produce PET. In an embodiment, the PTA polymerization unit 165 may comprise continuous polymerization of one or both of the purified terephthalic acid and DMT.

The continuous process for the formation of polyethylene terephthalate polyester is generally conducted in two stages. The first is the esterification stage in which one or both of the purified terephthalic acid and the DMT and the ethylene glycol react to form low modular weight oligomers and water. In general, a continuous feed of raw materials is used employing a molar ratio of ethylene glycol to terephthalic acid of from about 1 to about 1.6. The continuous feed enters a direct esterification vessel which is operated at a temperature of from about 240° C. to about 290° C. and at a pressure of from about 5 to about 85 psia for about 1 to about 5 hours. The reaction is typically uncatalyzed and forms low molecular weight oligomers and water. The water is removed as the esterification reaction proceeds and excess ethylene glycol is provided to enable the reaction to go to completion.

The second stage of the continuous process is the polycondensation stage in which the low molecular weight oligomers are polymerized to form PET polyester. The polycondensation stage generally employs a series of 2 or more vessels and is operated at a temperature of from about 250° C. to about 305° C. for about 1 to about 4 hours. Typically, the polycondensation reaction begins in a first vessel called the low polymerizer which is operated at a pressure range of from about 0 to about 70 mm of Hg. In the low polymerizer, the monomer polycondenses to form polyethylene terephthalate and ethylene glycol. The ethylene glycol is removed from the polymer melt using an applied vacuum to enable the polycondensation reaction to go to completion. The polymer melt is typically agitated to allow the ethylene glycol to escape from the polymer melt and be removed using the vacuum. In addition, the agitator generally aids the highly viscous polymer melt in moving through the polymerization vessel.

As the polymer melt is fed into successive vessels, the molecular weight and thus the intrinsic viscosity of the polymer melt increases. The temperature of each vessel is generally increased and the pressure decreased to allow greater polymerization in each successive vessel. The final vessel is generally called the high polymerizer and is operated at a pressure of from about 0 to about 40 mm Hg. As with the low polymerizer, each of the polymerization vessels communicates with a flash vessel and is typically agitated to facilitate the removal of ethylene glycol thus enabling the polycondensation reaction to go to completion. The retention time in the polymerization vessels and the feed rate of the ethylene glycol and one or both of the purified terephthalic acid and the DMT into the continuous process are determined in part based on the target molecular weight of the PET polyester. Because the molecular weight can be readily determined based on the intrinsic viscosity of the polymer melt, the intrinsic viscosity of the polymer melt is generally used to determine the feed rate of the reactants and the retention time in the polymerization vessels.

Once the polymer melt exits the polycondensation stage, typically from the high polymerizer, it is generally filtered and then extruded into polyester sheets, filaments, or pellets. Preferably, the polymer melt is extruded shortly after exiting the polycondensation stage and typically is extruded immediately after exiting the polycondensation stage. Once the PET polyester is extruded it is quenched, preferably in a water trough, to quickly decrease its temperature thus solidifying it. The solidified PET polyester is formed into pellets or cut into chips for storage and handling purposes. The solidified PET polyester is discharged from the PTA polymerization unit 165 in a product line 166.

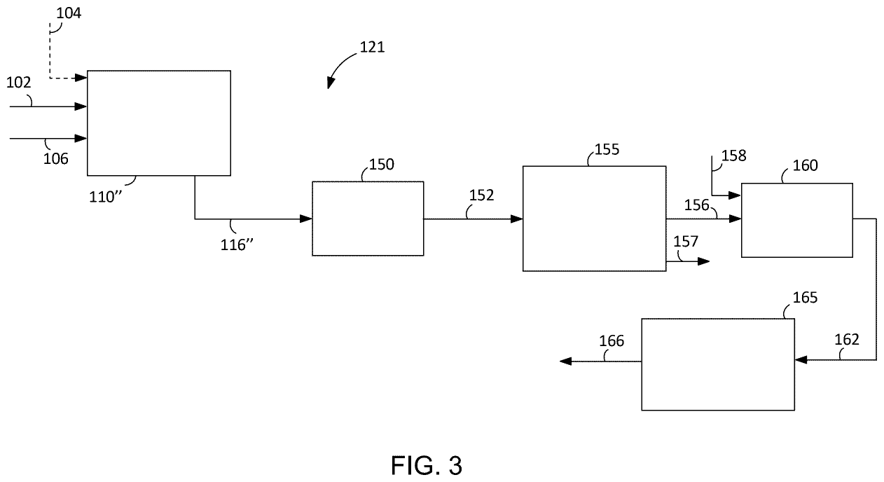

Another exemplary embodiment of the process for producing monomers 111 is shown in FIG. 3. Elements in FIG. 3 with the same configuration as in FIG. 2 will have the same reference numeral as in FIG. 2. Elements in FIG. 3 which have a different configuration as the corresponding element in FIG. 2 will have the same reference numeral but designated with a double prime symbol (″). ‘The configuration and operation of the embodiment of FIG. 3 is essentially the same as in FIG. 2 with the following exceptions.

In the embodiment shown in FIG. 3, the hydrocracking unit 110″ producing a heavy naphtha stream. The hydrocracked effluent stream may be fractionated in the fractionation column to produce a heavy naphtha stream comprising C₇₊ hydrocarbons, preferably C₇-C₁₀ hydrocarbons. The heavy naphtha stream is discharged from the hydrocracking unit 110″ in line 116″. In the embodiment as shown in FIG. 3, only the heavy naphtha stream is produced and taken from the fractionation column. The heavy naphtha stream in line 116″ is fed to the reforming unit 150 and reformed as previously described in FIG. 2. Rest of the process of same as earlier described in FIG. 2.

### EXAMPLE

A yield estimation study was performed for the process shown in FIG. 1. The results of the study are provided in Table 2 below. The hydrogen feed was ratioed to the fresh feed. The products are ratioed to the total feed to show product total as 1000% as below:

TABLE 2

| | | | New Case-Recycle | | |
|---|---|---|---|---|---|
| | Base-No Recycle | | of stream 144 | | |
| | | wt % of | | wt % of | Increase |
| | kmta | fresh feed | kmta | fresh feed | (%) |
| Fresh | 3,867 | 1.000 | 3,867 | 1.000 | |
| Hydrocarbon Feed | | | | | |
| to hydrocracking | | | | | |
| unit | | | | | |
| H2 for | 203 | 0.053 | 210 | 0.054 | |
| hydrocracking | | | | | |
| unit | | | | | |
| H2 for NEP unit | 191 | 0.049 | 198 | 0.051 | |
| Total Feed | 4,261 | | 4,275 | | |
| | | wt % of | | wt % of | |
| Total Products | kmta | total feed | kmta | total feed | |
| Hydrocracker LE | 70 | 0.016 | 72 | 0.017 | |
| Hydrogen (out) | 209 | 0.049 | 216 | 0.051 | |
| Fuel Gas | 806 | 0.189 | 835 | 0.195 | |
| Ethylene | 1,755 | 0.412 | 1,819 | 0.425 | 3.6% |
| Propylene | 960 | 0.225 | 994 | 0.233 | 3.5% |
| Crude C4s (w/ BD) | 92 | 0.022 | | 0.0 | |
| Pygas (no di- | 33 | 0.008 | | 0.0 | |
| olefins) | | | | | |
| Pyoil | 9 | 0.002 | | 0.0 | |
| Aromatics | 328 | 0.077 | 339 | 0.079 | 3.4% |
| Total Products | 4,261 | | 4,275 | | |

The results as shown in Table 2 above demonstrate increase in the yield of monomers over the base case.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process for producing monomers, comprising hydrocracking a pyrolysis oil stream in a hydrocracking reactor over a hydrocracking catalyst in the presence of a hydrocracking hydrogen stream at hydrocracking conditions to open rings present in the pyrolysis oil stream to produce a net hydrocracked effluent stream; fractionating the net hydrocracked effluent stream in a fractionation column to provide a naphtha stream; and contacting the naphtha stream with a NEP catalyst and hydrogen to produce a light paraffinic stream or with a reforming catalyst to produce an aromatics stream. An embodiment of the present disclosure is one, any or all of prior embodiments in this paragraph up through the first embodiment in this paragraph further comprising taking a C3-hydrocarbon stream from the fractionation column; and dehydrogenating the C3-hydrocarbon stream in a dehydrogenation reactor to produce a propylene stream. An embodiment of the present disclosure is one, any or all of prior embodiments in this paragraph up through the first embodiment in this paragraph further comprising separating the light paraffinic stream to provide an ethane stream and a propane stream; and cracking the ethane stream to produce an ethylene stream and a C₃₊ hydrocarbons stream. An embodiment of the present disclosure is one, any or all of prior embodiments in this paragraph up through the first embodiment in this paragraph further comprising recycling the C₃₊ hydrocarbons stream to the hydrocracking reactor. An embodiment of the present disclosure is one, any or all of prior embodiments in this paragraph up through the first embodiment in this paragraph further comprising combining the C₃₊ hydrocarbons stream to the hydrocracking reactor with the hydrocarbon feed stream to provide a charge stream, and hydrocracking the charge stream in the hydrocracking reactor to produce the hydrocracked effluent stream. An embodiment of the present disclosure is one, any or all of prior embodiments in this paragraph up through the first embodiment in this paragraph further comprising dehydrogenating the propane stream to produce a propylene stream. An embodiment of the present disclosure is one, any or all of prior embodiments in this paragraph up through the first embodiment in this paragraph, wherein the pyrolysis oil stream is produced from a plastic feed stream. An embodiment of the present disclosure is one, any or all of prior embodiments in this paragraph up through the first embodiment in this paragraph further comprising taking a heavy naphtha stream from the fractionation column; and contacting the heavy naphtha stream with the reforming catalyst to produce a reformed stream comprising an increased concentration of aromatics. An embodiment of the present disclosure is one, any or all of prior embodiments in this paragraph up through the first embodiment in this paragraph further comprising taking a light naphtha stream from the fractionation column; and charging the light naphtha stream to a NEP reactor for contacting with the NEP catalyst. An embodiment of the present disclosure is one, any or all of prior embodiments in this paragraph up through the first embodiment in this paragraph further comprising charging the reformed stream to an aromatic complex to produce a concentrated xylene stream, the aromatics complex comprising at least one of an aromatics extraction unit, a BTX fractionation zone, a xylene fractionation zone, a transalkylation unit, and an isomerization unit; and extracting paraxylene from the concentrated xylene stream to produce a paraxylene stream. An embodiment of the present disclosure is one, any or all of prior embodiments in this paragraph up through the first embodiment in this paragraph further comprising oxidizing the paraxylene stream to produce a terephthalic acid stream; and purifying the terephthalic acid stream to produce a purified terephthalic acid stream. An embodiment of the present disclosure is one, any or all of prior embodiments in this paragraph up through the first embodiment in this paragraph further comprising reacting the purified terephthalic acid stream with a glycol stream to produce polyethylene terephthalate monomer. An embodiment of the present disclosure is one, any or all of prior embodiments in this paragraph up through the first embodiment in this paragraph, wherein the hydrocracking reactor is a two stage hydrocracking reactor comprising a first stage hydrocracking reactor upstream of fractionation column and a second stage hydrocracking reactor downstream of fractionation column. An embodiment of the present disclosure is one, any or all of prior embodiments in this paragraph up through the first embodiment in this paragraph further comprising passing a petroleum stream to the hydrocracking reactor.

A second embodiment of the present disclosure is a process for producing monomers, comprising hydrocracking a pyrolysis oil stream in a hydrocracking reactor over a hydrocracking catalyst in the presence of a hydrocracking hydrogen stream at hydrocracking conditions to open rings present in the pyrolysis oil stream to produce a net hydrocracked effluent stream; fractionating the net hydrocracked effluent stream in a fractionation column to provide a C₃₋ hydrocarbon stream and a naphtha stream; contacting the naphtha stream with a NEP catalyst and hydrogen to produce a light paraffinic stream or with a reforming catalyst to produce an aromatics stream; and dehydrogenating the C3-hydrocarbon stream in a dehydrogenation reactor to produce a propylene stream. An embodiment of the present disclosure is one, any or all of prior embodiments in this paragraph up through the second embodiment in this paragraph further comprising separating the light paraffinic stream to provide an ethane stream and a propane stream; and cracking the ethane stream to produce an ethylene stream. An embodiment of the present disclosure is one, any or all of prior embodiments in this paragraph up through the second embodiment in this paragraph, wherein the pyrolysis oil stream is produced from a plastic feed stream. An embodiment of the present disclosure is one, any or all of prior embodiments in this paragraph up through the second embodiment in this paragraph further comprising fractionating the net hydrocracked effluent stream to provide a light naphtha stream and a heavy naphtha stream; contacting the light naphtha stream with the NEP catalyst; and contacting the heavy naphtha stream with reforming catalyst.

A third embodiment of the present disclosure is a process for producing monomers, comprising hydrocracking a pyrolysis oil stream in a two stage hydrocracking reactor over a hydrocracking catalyst in the presence of a hydrocracking hydrogen stream at hydrocracking conditions to open rings present in the pyrolysis oil stream to produce a net hydrocracked effluent stream; fractionating the net hydrocracked effluent stream in a fractionation column to provide a naphtha stream; and contacting the naphtha stream with a NEP catalyst and hydrogen to produce a light paraffinic stream or with a reforming catalyst to produce an aromatics stream. An embodiment of the present disclosure is one, any or all of prior embodiments in this paragraph up through the third embodiment in this paragraph, wherein the pyrolysis oil stream is produced from a plastic feed stream. An embodiment of the present disclosure is one, any or all of prior embodiments in this paragraph up through the third embodiment in this paragraph further comprising separating the light paraffinic stream to provide an ethane stream and a propane stream; and cracking the ethane stream to produce an ethylene stream. An embodiment of the present disclosure is one, any or all of prior embodiments in this paragraph up through the third embodiment in this paragraph further comprising dehydrogenating the propane stream to produce a propylene stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing monomers, comprising:
hydrocracking a hydrocarbon feed stream in a hydrocracking reactor over a hydrocracking catalyst in the presence of a hydrocracking hydrogen stream at hydrocracking conditions to open rings present in said hydrocarbon feed stream to produce a hydrocracked effluent stream;
fractionating said hydrocracked effluent stream in a fractionation column to provide a naphtha stream; and
contacting said naphtha stream with a NEP catalyst and hydrogen to produce a light paraffinic stream and/or with a reforming catalyst to produce an aromatics stream.

2. The process of claim 1 further comprising:
taking a C₃₋ hydrocarbon stream from the fractionation column; and
dehydrogenating said C₃₋ hydrocarbon stream in a dehydrogenation reactor to produce a propylene stream.

3. The process of claim 1 further comprising:
separating said light paraffinic stream to provide an ethane stream and a propane stream; and
cracking said ethane stream to produce an ethylene stream and a C₃₊ hydrocarbons stream.

4. The process of claim 3 further comprising recycling said C₃₊ hydrocarbons stream to the hydrocracking reactor.

5. The process of claim 4 further comprising:
combining said C₃₊ hydrocarbons stream to the hydrocracking reactor with said hydrocarbon feed stream to provide a charge stream; and
hydrocracking said charge stream in the hydrocracking reactor to produce said hydrocracked effluent stream.

6. The process of claim 3 further comprising dehydrogenating said propane stream to produce a propylene stream.

7. The process of claim 1, wherein said hydrocarbon feed stream comprises at least one of a diesel stream, a vacuum gas oil stream, distillate range hydrocarbons, and a pyrolysis oil stream.

8. The process of claim 1 further comprising:
taking a heavy naphtha stream from the fractionation column; and
contacting said heavy naphtha stream with the reforming catalyst to produce a reformed stream comprising an increased concentration of aromatics.

9. The process of claim 1 further comprising:
taking a light naphtha stream from the fractionation column; and
charging said light naphtha stream to a NEP reactor for contacting with the NEP catalyst.

10. The process of claim 8 further comprising:
charging said reformed stream to an aromatic complex to produce a xylene containing stream, the aromatics complex comprising at least one of an aromatics extraction unit, a BTX fractionation zone, a xylene fractionation zone, a transalkylation unit, and an isomerization unit; and
extracting paraxylene from said xylene containing stream to produce a paraxylene stream.

11. The process of claim 10 further comprising:
oxidizing said paraxylene stream to produce an oxidized stream comprising at least one of a terephthalic acid stream and a dimethyl terephthalate stream; and
purifying said oxidized stream to produce at least one of a purified terephthalic acid stream and a purified dimethyl terephthalate stream.

12. The process of claim 11 further comprising reacting at least one of said purified terephthalic acid stream and said purified dimethyl terephthalate stream with a glycol stream to produce polyethylene terephthalate monomer.

13. The process of claim 1, wherein the hydrocracking reactor is a two stage hydrocracking reactor comprising a first stage hydrocracking reactor upstream of fractionation column and a second stage hydrocracking reactor downstream of fractionation column.

14. The process of claim 1 further comprising passing a petroleum stream to the hydrocracking reactor.

15. A process for producing monomers, comprising:
hydrocracking a hydrocarbon feed stream in a hydrocracking reactor over a hydrocracking catalyst in the presence of a hydrocracking hydrogen stream at hydrocracking conditions to open rings present in said hydrocarbon feed stream to produce a hydrocracked effluent stream;
fractionating said hydrocracked effluent stream in a fractionation column to provide a C₃₋ hydrocarbon stream and a naphtha stream;
contacting said naphtha stream with a NEP catalyst and hydrogen to produce a light paraffinic stream or with a reforming catalyst to produce an aromatics stream, or
contacting said naphtha stream with the NEP catalyst and hydrogen to produce said light paraffinic stream and with the reforming catalyst to produce said aromatics stream; and
dehydrogenating said C₃₋ hydrocarbon stream in a dehydrogenation reactor to produce a propylene stream.

16. The process of claim 15 further comprising:
separating said light paraffinic stream to provide an ethane stream and a propane stream; and
cracking said ethane stream to produce an ethylene stream.

17. The process of claim 15, wherein said hydrocarbon feed stream comprises at least one of a diesel stream, a vacuum gas oil stream, distillate range hydrocarbons, and a pyrolysis oil stream.

18. The process of claim 15 further comprising:
charging said aromatics stream to an aromatic complex to produce a xylene containing stream, the aromatics complex comprising at least one of an aromatics extraction unit, a BTX fractionation zone, a xylene fractionation zone, a transalkylation unit, and an isomerization unit; and
extracting paraxylene from said xylene containing stream to produce a paraxylene stream.

19. A process for producing monomers, comprising:
hydrocracking a hydrocarbon feed stream in a hydrocracking reactor over a hydrocracking catalyst in the presence of a hydrocracking hydrogen stream at hydrocracking conditions to open rings present in said hydrocarbon feed stream to produce a hydrocracked effluent stream;
fractionating said hydrocracked effluent stream in a fractionation column to provide a naphtha stream;
contacting said naphtha stream with a NEP catalyst and hydrogen to produce a light paraffinic stream or with a reforming catalyst to produce an aromatics stream,
contacting said naphtha stream with the NEP catalyst and hydrogen to produce said light paraffinic stream and with the reforming catalyst to produce said aromatics stream; and
producing at least one of olefinic monomers from said light paraffinic stream and polyethylene terephthalate monomer from said aromatics stream.

20. The process of claim 19, wherein said hydrocarbon feed stream comprises at least one of a diesel stream, a vacuum gas oil stream, distillate range hydrocarbons, and a pyrolysis oil stream.

21. The process of claim 19 further comprising:
separating said light paraffinic stream to provide an ethane stream and a propane stream; and
cracking said ethane stream to produce an ethylene stream.

22. The process of claim 21 further comprising dehydrogenating said propane stream to produce a propylene stream.
